(19) United States
(12) Patent Application Publication
(10) Pub. No.: US 2007/0152171 A1
(43) Pub. Date: Jul. 5, 2007

(54) Free electron laser

(76) Inventors: Michael Goldstein, Ridgefield, CT (US); David L. Williams, San Jose, CA (US)

Correspondence Address: Stephen M. De Klerk;BLAKELY, SOKOLOFF, TAYLOR & ZAFMAN LLP, Seventh Floor, 12400 Wilshire Boulevard, Los Angeles, CA, 90025, US

(21) Appl. No.: 11/322,899

(22) Filed: Dec. 30, 2005

(51) Int. Cl. A61N 5/00

(52) U.S. Cl. 250/492.3

(57) ABSTRACT

A free electron laser is disclosed. The free electron laser separates pulse bunching at a first electron energy from light generation stage at a second electron energy. A first wiggler pulse bunches the electrons and a second wiggler generates light. The first wiggler may be an optical buncher with an injected seed wave, and the second wiggler can be a magnetic wiggler, optical wiggler, resonant transition radiator, parametric radiation radiator, Cerenkov radiation radiator or a Smith-Purcell radiation radiator. The disclosed free electron laser is particularly useful for lithography applications at an extreme ultraviolet wavelength range near 13.5 nm.

### FIELD OF THE INVENTION

The present invention relates to the field of free electron lasers and, in particular, to an apparatus and method for attaining extreme ultraviolet (EUV) wavelengths.

### BACKGROUND OF THE INVENTION

Many every day items, including computers, phones, and even our cars include computer chips. Programs run on computer chips, providing these items with electronic functionality. These programs require complex integrated circuits (IC's) to operate. These circuits are built in layers on a silicon wafer using chemicals, gases and light. A layer of silicon oxynitride is grown on the silicon wafer and a resist is deposited on the wafer. In a photolithography process, UV light is passed through a patterned mask (or stencil) onto the resist-coated wafer. The light reacts with the resist, leaving features of the IC on the wafer. The unexposed areas (resist and silicon dioxide) are removed. This process is repeated several times to form several layers of circuit features. Ion implantation is used to expose areas of the wafer with ions, altering the way the wafer conducts electricity. Electrical connections are added to the structure, and a protective package is provided to form the completed computer chip.

In order to keep pace with the demand for the printing of ever smaller features of ICs in the semiconductor device field, lithography tool manufacturers have found it necessary to gradually reduce the wavelength of the light used for imaging and to design imaging systems with ever larger numerical apertures. In order to scale beyond the 32 nm feature size node, extreme ultraviolet (EUV) wavelength light (i.e., 5-20 nm) will be required in lithography imaging systems.

Existing EUV sources are plasma based. These existing sources are capable of producing the desired wavelengths; however, producing the desired wavelengths using the plasma based sources has several disadvantages, including production of debris that can contaminate and damage the optics used in EUV lithography systems. In addition, these sources emit radiation in all directions and, therefore, have poor etendue. These sources also emit a broad spectral range that must then be filtered to obtain EUV light. Moreover, the EUV light that is produced by the plasma based sources is low power EUV.

In existing free electron lasers, an electron gun emits an electron beam, which is then accelerated using a linear accelerator to a high energy. The high energy electron beam then passes through an undulator or wiggler and emits radiation in the EUV wavelength range. Thus, pulse bunching and light generation take place together. However, electrons are more massive at high energy due to relativistic effects, and are therefore difficult to wiggle. As a result, these existing free electron lasers are expensive, large and cumbersome for obtaining EUV light.

### BRIEF DESCRIPTION OF THE DRAWINGS

The invention is described by way of example with reference to the accompanying drawings, wherein:

FIG. 1 is a perspective view of an extreme ultraviolet (EUV) lithography system;

FIG. 2 is a cross-sectional side view of an EUV source, used in the system of FIG. 1, according to an embodiment of the invention;

FIGS. 3A and 3B are side views of electron interaction with a radiation field leading to pulse-bunching;

FIG. 4 is a perspective view of a magnetic wiggler that can form part of the EUV source of FIG. 2, according to one embodiment of the invention;

FIG. 5 is a side view of a laser wiggler that can form part of the EUV source of FIG. 2, according to another embodiment of the invention;

FIG. 6 is a side view of a laser accelerator that can form part of the EUV source of FIG. 2, according to a further embodiment of the invention;

FIG. 7 is a cross-sectional side view of an element of a transition radiation stack, forming part of the EUV source of FIG. 2 according to one embodiment of the invention;

FIG. 8 is a side view of an EUV source according to another embodiment of the invention;

FIG. 9 is a side view of an EUV source according to a further embodiment of the invention;

FIG. 10 is a side view of an EUV source according to yet a further embodiment of the invention;

FIG. 11 is a perspective view of Compton scattering that can be used with the EUV source of FIG. 10 according to an embodiment of the invention; and

FIG. 12 is a perspective view of an EUV source according to yet a further embodiment of the invention.

### DETAILED DESCRIPTION

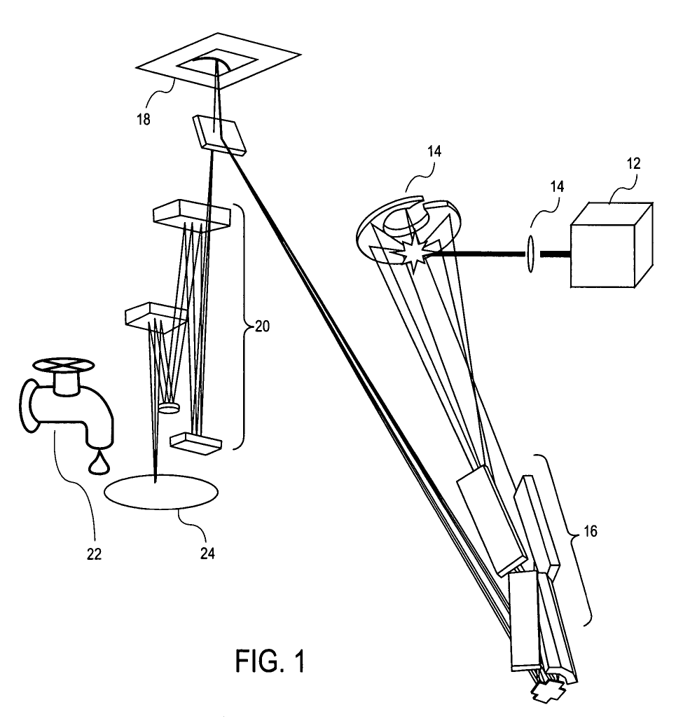

FIG. 1 illustrates an extreme ultraviolet (EUV) lithography system 10, according to an embodiment of the invention, which can be used in the manufacture of integrated circuits (ICs). The lithography system 10 includes an EUV light source 12, collector optics 14, an illuminator 16, a reflective mask 18, reflective reduction optics 20 and a resist source 22, for the purposes of making lithography patterns on a wafer 24.

The light source 12 is an EUV radiation light source (i.e., light source 12 radiates light in the 5-20 nm wavelength range). Ideally, the light source 12 produces EUV radiation having an approximately 13.5 nm wavelength. EUV radiation is referred to throughout the specification; however, it will be appreciated by those of skill in the art that EUV radiation also refers to EUV light.

The collector optics 14 are arranged to collect the EUV radiation from the light source 12 and direct the EUV radiation toward the illuminator 16. The collector optics 14 illustrated is a ring-shaped mirror.

The illuminator 16 is arranged to gather the EUV radiation from the collector optics 14 and focus and direct the EUV radiation on the mask 18. The illuminator 16 controls the uniformity and intensity of the EUV radiation that is directed to the mask 18.

The mask 18 is arranged to receive the EUV radiation from the illuminator 16. The mask 18 has a reflective pattern, corresponding to the IC features desired on the wafer 24. The pattern includes a patterned absorber of EUV radiation placed on top of a multi-layer reflector deposited on a substrate. The reflectance spectrum of the mask 18 is matched to that of the reduction optics 20. The mask 18 is arranged to direct the patterned light reflected from the mask 18 toward the reduction optics 20 and eventually on to the wafer 24.

The reduction optics 20 are arranged to project the patterned light from the mask 18 onto wafer 24. The reduction optics 20 shown include four curved mirrors, which have a reduction factor of 4. These mirrors typically include a multilayer coating for reflecting the EUV radiation, which may be Mo/Si, Mo/Be, or the like.

The resist source 22 provides a resist of an EUV radiation-sensitive material. The resist is a chemical that hardens when exposed to the EUV radiation. The resist is applied to the wafer 24, such that when the hardened resist is schematically etched, some of the resist is removed from the wafer, and the pattern from the mask 18 is left on the wafer 24 by the remaining resist.

The wafer 24 is a semiconductor wafer. The wafer 24 is coated with resist from the resist source 22. The resist-coated wafer 24 is aligned with the mask 18 and the reduction optics 20 to receive the patterned EUV radiation. The hardened resist is etched using a chemical treatment, leaving a layer of metal in the shape of the pattern from the mask on the wafer 24. The mask 18 and the wafer 24 are simultaneously scanned in opposite directions, with the mask moving four times faster than the wafer to scan features of the ICs onto the wafer 24.

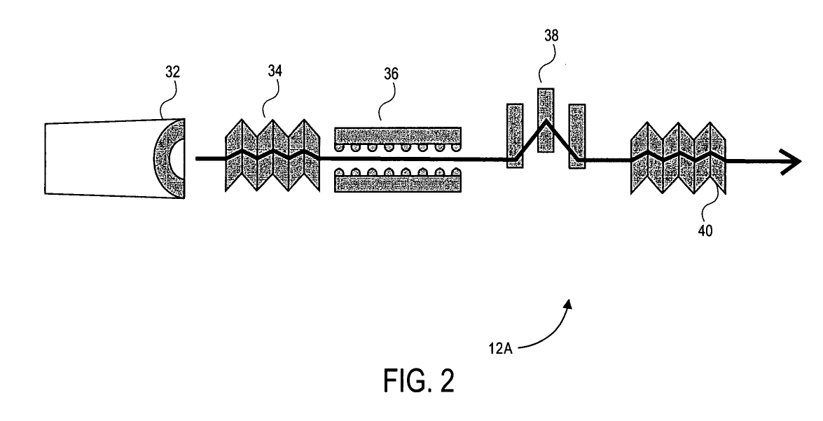

According to an embodiment of the present invention, a free electron laser capable of emitting extreme ultraviolet radiation is shown in FIG. 2. The free electron laser 12A may be used as the light source in a lithography system, such as the light source 12 in the lithography system 10.

The free electron laser 12A includes an electron gun 32, a buncher 34, an accelerator 36, an electron dispersion compensator 38 and an EUV emitter 40. Each of the electron gun 32, buncher 34, accelerator 36, electron dispersion compensator 38 and EUV emitter 40 are aligned with one another.

The electron gun 32 emits a beam of electrons (i.e., electron beam). The electron gun 32 typically includes a thermionic cathode or photo-cathode and an accelerating field for emitting the electron beam. Ideally, the power of the electron beam generated is any value up to about 100 keV.

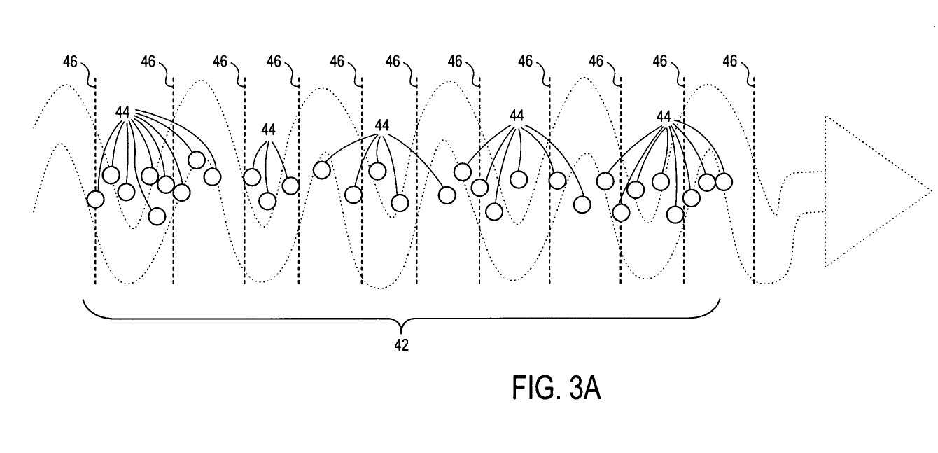

The buncher 34 is aligned with the electron gun 32, and pulse bunches and/or micro bunches electrons in the electron beam. FIGS. 3A and 3B illustrate bunch generation. FIG. 3A shows an electron beam 42 having a plurality of electrons 44. A radiation field 46 is generated and directed toward the electron beam 42. As the electrons 44 interact with the radiation field 46, the electron beam 42 modulates to the radiation wavelength. The electrons 44 tend to drift towards phases of the radiation field and bunch with other nearby electrons with the periodicity of the radiation field to form bunches 48, as shown in FIG. 3B. The buncher thus modulates the density of the electrons in the electron beam. The radiation field, which leads to bunching, is generated by a wiggler or an undulator.

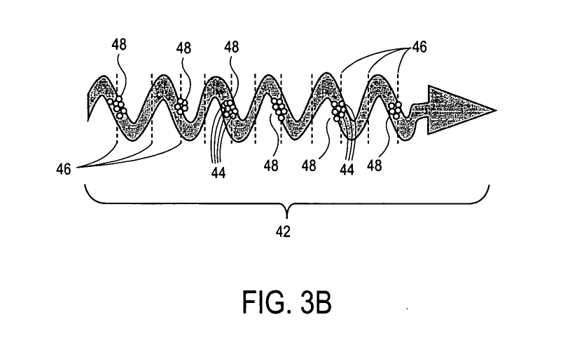

FIG. 4 illustrates a magnetic wiggler, which can be used with any of the free electron lasers disclosed herein, according to an embodiment of the invention. The buncher 34 includes a plurality of north magnets 52 and south magnets 54. The magnets 52, 54 generate a radiation field. The electron beam 44 interacts with the radiation field created by the magnets 52, 54 to generate bunching, as described above with reference to FIGS. 3A and 3B.

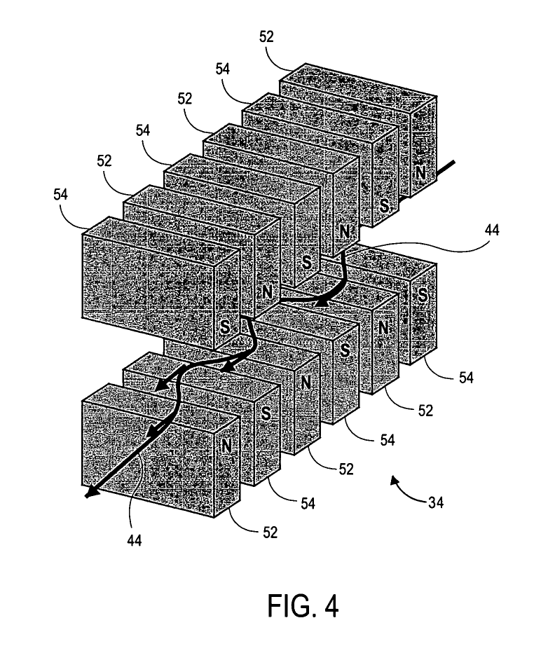

FIG. 5 illustrates an optical wiggler, which can be used with any of the free electron lasers disclosed herein, according to a further embodiment of the invention. The buncher 34 illustrated includes a laser 60. The laser 60 produces laser light 62 which creates a radiation field. The radiation field is injected into the path of the electron beam. The injected laser light is polarized. The injected laser light and its associated radiation field can be slowed if injected at an angle. The electron beam 44 interacts with the field generated by the laser 60 to generate bunching, as described above with reference to FIGS. 3A and 3B.

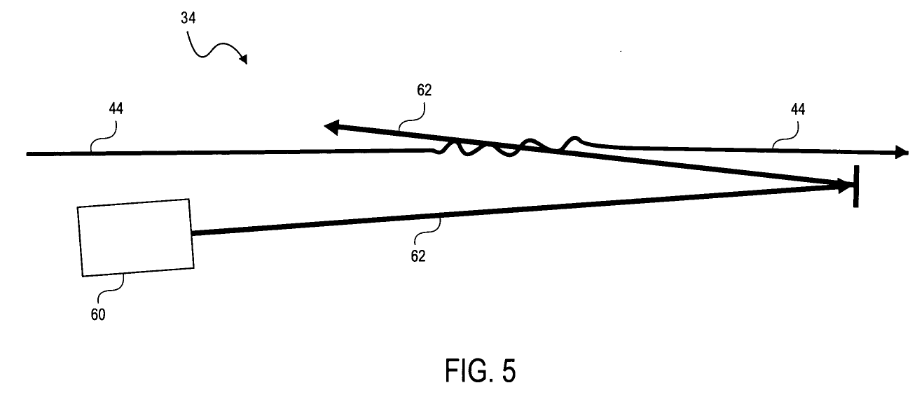

With reference back to FIG. 2, the accelerator 36 is aligned with the buncher 34 and accelerates the bunched electrons. FIG. 6 illustrates a linear accelerator, which can be used with any of the free electron lasers disclosed herein, according to an embodiment of the invention. The illustrated accelerator 36 is a laser accelerator. The accelerator 36 includes a laser (not shown) and a lens 74 having an opening 76 therein. The laser generates laser light 72. The laser light 72 passes through the lens 74, while the electron beam 44 passes through the opening 76 in the lens 74. The lens 74 focuses the laser light 72, thereby accelerating the laser light 72 and its associated electric field. The electrons 44 in the electron beam 42 are carried and accelerated by the electric field of light created by the laser 70.

With reference back to FIG. 2, the electron dispersion compensator 38 is aligned with the accelerator 36 and further micro-bunches the bunched electrons. Often, when electrons are accelerated, the electrons within each electron bunch are dispersed. By passing the bunched electrons through the electron dispersion compensator 38, electrons within each bunch are further bunched, thereby correcting gross mismatch in electron displacement in each bunch. The illustrated electron dispersion compensator 38 is a chicane of magnets.

The EUV emitter 40 is aligned with the electron dispersion compensator 38 and generates EUV radiation. The EUV emitter 40 extracts EUV light (i.e., 5-20 nm) from the bunched electrons and radiates the light. In one embodiment, the EUV emitter generates EUV light having approximately a 13.5 nm wavelength. The EUV emitter 38 may be a magnetic wiggler or an optical wiggler, as previously described with reference to FIGS. 4 and 5. Alternatively, the EUV emitter 40 uses resonant transition radiation, Cerenkov radiation, Smith-Purcell radiation, or parametric radiation to generate EUV radiation.

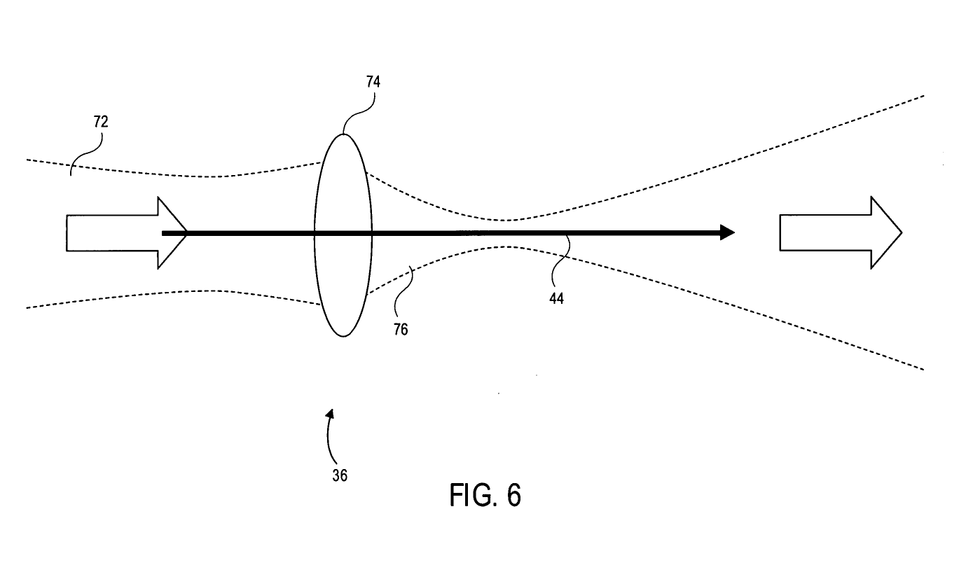

FIG. 7 illustrates an EUV emitter, which can be used with any of the free electron lasers disclosed herein, according to an embodiment of the invention. The EUV emitter illustrated is a transition radiation stack 80 for generating transition radiation. The transition radiation stack 80 includes a dielectric interface 82 and tri-layer foil stacks 84. The opening between the stacks 84 creates a vacuum 86. In use, a plurality of stacks 80 are aligned to create a plurality of dielectric interfaces 82, and are spaced such that the peaks and troughs of the electric field from the interfaces 82 are aligned. The electron beam passes through the vacuum 86 and dielectric interface 82. Emission of a photon occurs at the transition between two media of differing dielectric constants (i.e., the dielectric interface 82 and vacuum 86) and occurs as a result of an induced polarization in the dielectric interface 82.

With reference back to FIG. 2, in use, the electron gun 32 emits an electron beam. The electrons are bunched at the buncher 34, and a bunched electron beam is emitted from the buncher 34. The bunched electrons are accelerated in the accelerator 36. The bunched electrons are further bunched in the electron dispersion compensator 38. EUV radiation is extracted from the accelerated bunched electrons at the EUV emitter 40.

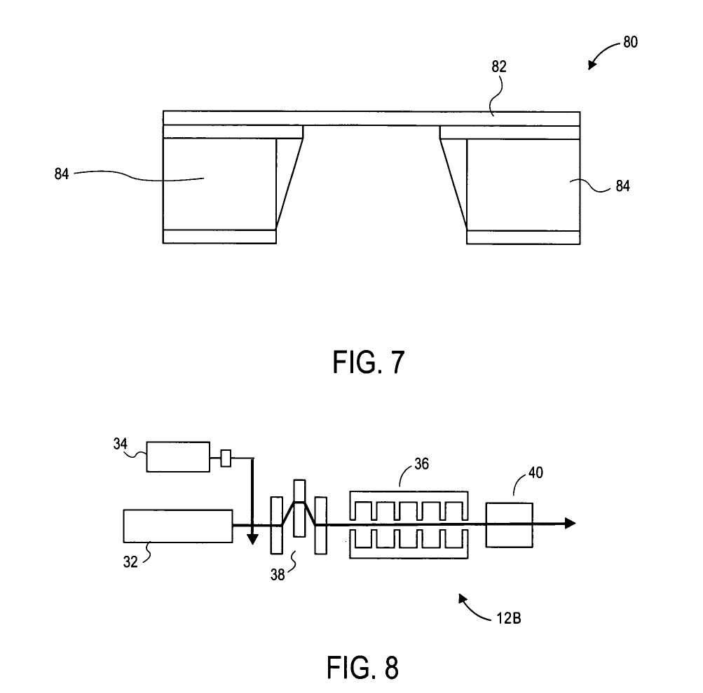

According to an embodiment of the present invention, a free electron laser capable of emitting extreme ultraviolet radiation is shown in FIG. 8. The free electron laser 12B may be used as a light source in a lithography system, such as the light source 12 in the lithography system 10.

The free electron laser 12B includes an electron gun 32, a buncher 34, an electron dispersion compensator 38, an accelerator 36 and an EUV emitter 40. The free electron laser 12B differs from the free electron laser 12A in that the electron dispersion compensator 38 is arranged in between the buncher 34 and the accelerator 36, as opposed to between the accelerator 36 and the EUV emitter 40. As with the free electron laser 12A, each of the electron gun 32, buncher 34, electron dispersion compensator 38, accelerator 36, and EUV emitter 40 of the free electron laser 12B are aligned with one another.

In use, the electron gun 32 emits an electron beam, the beam having a plurality of electrons. The electrons are pulse bunched at the buncher 34, and a bunched electron beam is emitted from the buncher 34. The bunched electrons are then further bunched in the electron dispersion compensator 38. The bunched electron beam is then accelerated in the accelerator 36. The accelerated, bunched electrons are emitted as EUV light at the EUV emitter 40.

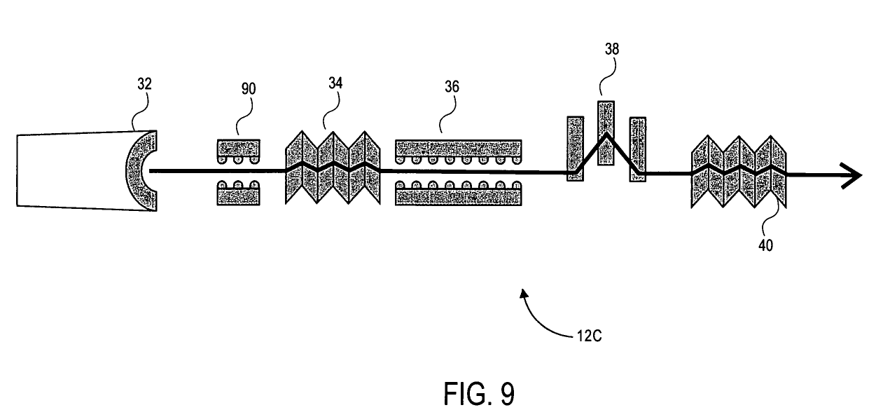

According to an embodiment of the present invention, a free electron laser capable of emitting extreme ultraviolet radiation is shown in FIG. 9. Free electron laser 12C may be used as a light source in a lithography system, such as the light source 12 in the lithography system 10.

Free electron laser 12C includes an electron gun 32, a pre-accelerator 90, a buncher 34, an accelerator 36, an electron dispersion compensator 38 and an EUV emitter 40. The free electron laser 12C differs from the free electron laser 12A in that the free electron laser 12C includes a pre-accelerator 90 before the buncher 34. As with the free electron laser 12A, each of the electron gun 32, pre-accelerator 90, buncher 34, accelerator 36, electron dispersion compensator 38 and EUV emitter 40 of the free electron laser 12C are aligned with one another.

In use, the electron gun 32 emits an electron beam, the beam having a plurality of electrons. The electrons are accelerated to at most 12 MeV at the pre-accelerator 90. It is known that electrons moving at 1 MeV move at 90% speed of light, and hence, are still influenced by an electric field moving in the same direction. The pre-accelerated electrons are thus bunched at the buncher 34, and a bunched electron beam is emitted from the buncher 34.

The bunched electrons are then accelerated again in accelerator 36 to a higher energy level. The bunched electrons are further bunched in the electron dispersion compensator 38. The twice accelerated, bunched electrons are emitted as EUV light at the EUV emitter 40.

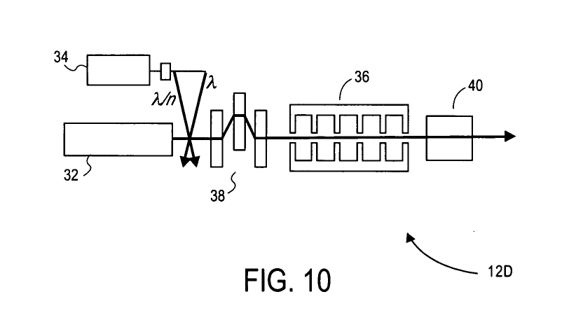

According to an embodiment of the present invention, a free electron laser capable of emitting extreme ultraviolet radiation is shown in FIG. 10. Free electron laser 12D may be used as a light source in a lithography system, such as the light source 12 in the lithography system 10.

The free electron laser 12D includes an electron gun 32, a buncher 34, an electron dispersion compensator 38, an accelerator 36 and an EUV emitter 40. The illustrated pulse buncher 34 is an optical wiggler having a frequency λ The free electron laser 12D differs from the previously disclosed free electron lasers (12A, 12B, 12C) in that the radiation field generated by the buncher 34 is directed at an oblique angle of incidence to the electron beam. By injecting the field at an oblique angle, a harmonic λ/n of the optical wiggler frequency λ is generated and injected into the buncher 34, as well.

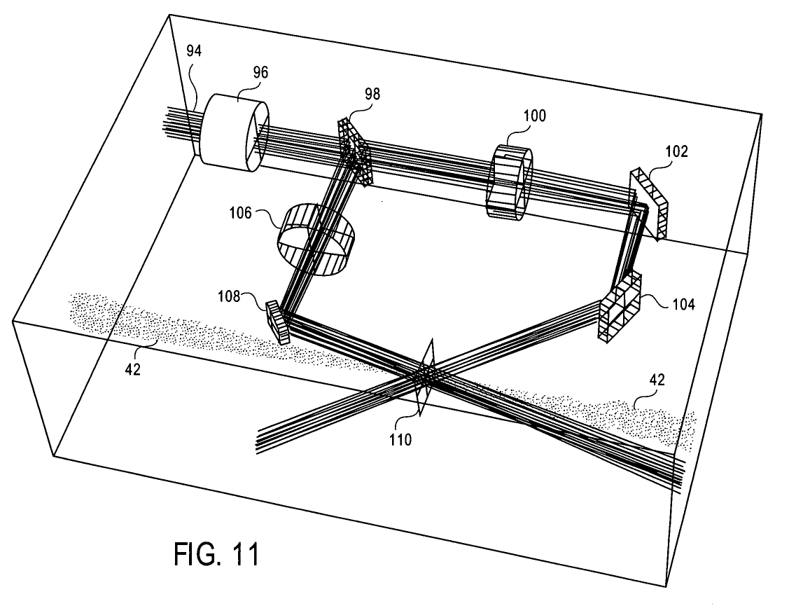

FIG. 11 is a detailed view of the buncher 34, illustrating Compton scattering, which leads to high gain harmonic pulse bunching generation, according to an embodiment of the invention. The buncher 34 illustrated is an optical wiggler. The optical wiggler 92 includes a laser 94, a crystal 96, a dichotic mirror 98, a green lens 100, green mirrors 102, 104, an ultraviolet lens 106 and an ultraviolet mirror 108. The illustrated laser 94 is a green laser, which is doubled by the crystal 96 to give both green and ultraviolet co-propagating light. The ultraviolet light is a back-scattered electromagnetic wave. The wavelengths are separated by the dichotic mirror 98 (i.e., a mirror that transmits green and reflects ultraviolet light). The green light is focused by the lens 100 and directed by the mirrors 102, 104 to intersect the electron beam 42 at an angle. The ultraviolet light is focused by the lens 106 and directed by the mirror 108 to intersect the electron beam 42 at an angle, as well. The ultraviolet light propagates in the same direction as the electron beam 42, while the green light propagates in the opposite direction of the electron beam 42. The electron beam and the two optical beams (i.e., green light and ultraviolet light) interact in a small volume, the centre of which is shown by the cross-heirs 110. An additional seed laser may be used to generate a third beam, which is seeded with the ultraviolet beam. In one embodiment, the laser wavelengths are, for example, green (532 nm) light and ultraviolet (266 nm) light, which are the 2^{nd} and 4^{th} harmonics, respectively, of a Nd:YAG laser. The angle of injection is dependent upon the power and frequency of the laser and electron beam, as appreciated by those of skill in the art.

The electron beam is therefore seeded with a harmonic of the wiggler wavelength (i.e., the Compton wavelength) and the wiggler wavelength. The electron beam is bunched at the Compton wavelength, as described above with reference to FIG. 3B. The electron bunches thus have a density variation having a harmonic component.

With reference back to FIG. 10, in use, the electron gun 32 emits an electron beam, the beam having a plurality of electrons. The electrons are bunched at the buncher 34. The electrons are bunched by injecting the field at an oblique angle of incidence, thereby injecting both the wiggler wavelength and the Compton wavelength (at a harmonic of the wiggler wavelength) into the buncher 34. A bunched electron beam is emitted from the buncher 34. The bunched electrons are then further bunched in the electron dispersion compensator 38. The bunched electron beam is then accelerated in the accelerator 36. The accelerated, bunched electrons are emitted as EUV radiation at the EUV emitter 40.

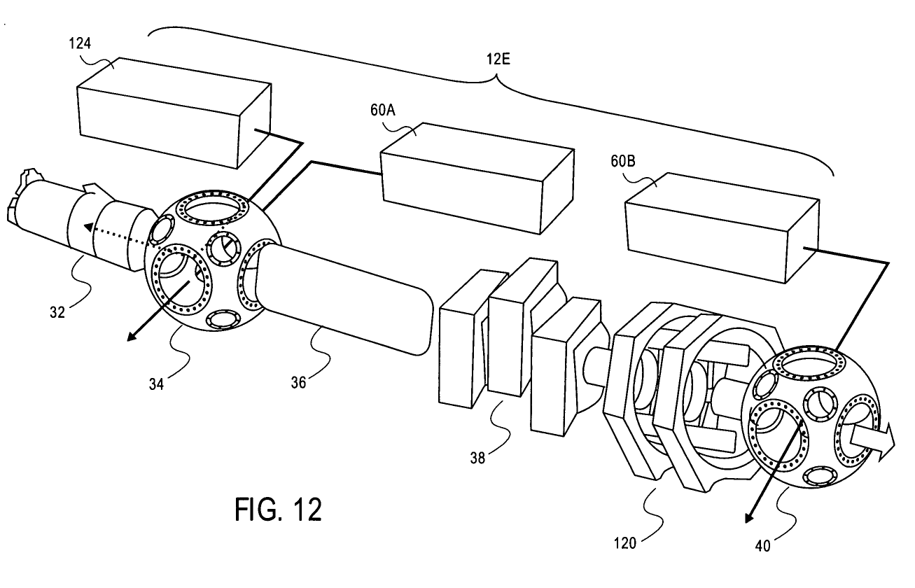

According to an embodiment of the present invention, a free electron laser capable of emitting extreme ultraviolet radiation is shown in FIG. 12. The free electron laser 12E may be used as a light source in a lithography system, such as the light source 12 in the lithography system 10.

The free electron laser 12E includes an electron gun 32, a buncher 34, an accelerator 36, an electron dispersion compensator 38, a focusing quadrupole 120, and an EUV emitter 40. The electron gun 32, buncher 34, accelerator 36, electron dispersion compensator 38, focusing quadrupole 120 and EUV emitter 40 are all aligned with one another. The focusing quadrupole is provided after the electron dispersion compensator 38 to focus the electron beam toward the EUV emitter 40.

The illustrated electron gun 32 includes a laser 124 for producing and accelerating the electron beam having the desired characteristics. The illustrated buncher 34 and the illustrated EUV emitter 40 are both optical devices. Lasers 60A and 60B inject a radiation field into the buncher 34 and EUV emitter 40, respectively, to generate pulse bunching and EUV radiation, respectively, as described above with reference to FIG. 11.

In use, the electron gun 32 emits an electron beam, the beam having a plurality of electrons. The electrons are bunched at the buncher 34, and a bunched electron beam is emitted from the buncher 34. The bunched electron beam is then accelerated in the accelerator 36. The accelerated bunched electron beam is then further bunched at the electron dispersion compensator 38 to compensate for electron dispersion. The focusing quadrupole 120 focuses the bunched beam toward the EUV emitter 40, which extracts EUV light from the beam.

Although it has not been explicitly discussed heretofore, those of skill in the art will recognize that the lithography process and EUV generation processes each desirably occur in a vacuum.

Although embodiments of the present invention have been described in relation to a lithography system, the free electron lasers described herein may be used in any system requiring EUV light, such as, for example, metrology.

An exemplary lithography system has been described herein. However, variations to the described lithography system are envisioned. Any patterning device may be used with the lithography system, such as masks, mirror arrays, programmable digital imaging systems and the like. Although the projection system has been described as using four curved mirrors, it is envisioned that any projection system may be used. For example, the projection system may use fewer or a greater number of mirrors, the projection system may have a different reduction factor, and the like.

Exemplary free electron lasers capable of producing EUV light have been disclosed herein. However, it is envisioned that various features of each of the embodiments may be combined with one another or altered, as will be obvious to those of skill in the art, so long as the bunching and light generation aspects of the process are separated by an acceleration process. For example, a free electron laser having a pre-accelerator may also use harmonic-gain pulse bunching, and the like.

It is also envisioned that the free electron laser disclosed herein may be used to generate any kind of light, such as, for example, soft x-ray radiation, ultraviolet radiation, and the like.

The components of the free electron lasers disclosed herein have been described as being aligned with one another. It will be appreciated that the components are therefore adjacent one another.

The buncher 34 has been described as bunching the electrons in the electron beam. The accelerator 32 may emit electrons in pulsed macro-bunches. In such a case, the buncher 34 is used to create micro-bunched pulses. Alternatively, the buncher 34 may both generate the pulse bunches and create micro-bunches.

If a field is injected at the pulse buncher at an angle to the electron beam 0, the speed in the direction of the electron beam is reduced by 1/cos θ, thereby allowing phase matching between the bunches and the field. Hence, it may be desirable to inject the field at an angle to the electron beam. In addition, although magnetic and optical wigglers have been disclosed herein, it is envisioned that electrical wigglers may also be used in embodiments of the invention.

Although the linear accelerator was illustrated as a laser accelerator, any accelerator, such as an RF accelerator, may be used.

The chicane may be any device capable of compensating for electron dispersion. Correction of electron divergence is desirable between the bunching and radiating sections of the system. Focusing lenses, such as a focusing quadrupole, may be needed before and/or after the electron dispersion compensator.

It will be appreciated that the optical wiggler disclosed in FIG. 11 may also be used as the EUV emitter. The EUV emitter may thus use Compton or Thompson back-scattered radiation to generate EUV. It is also envisioned that a seed laser does not need to be used if the Compton wave is sufficiently strong.

A transition radiation stack has been described herein. The dielectric interface may be any dielectric material, such as a metal film. In one embodiment, the metal film is a multilayer stack of molybdenum. In one embodiment, twenty layers of molybdenum are used in a system wherein the electron beam is at 9 MeV, 1 kHz and 0.7 mA when it arrives at the transition radiation stack. In one embodiment, a Si/Nb multilayer is fabricated on top of a thin Mo foil supported on Al. In another embodiment, the stack is formed of MoN/Mo/MoN. The temperature of the stack should be controlled to avoid buckling, as known to those of skill in the art.

Maximum extraction of light occurs when electrons emit coherently. It is advantageous to bunch the electrons within one quarter of the wavelength (λ) of the EUV period. Electrons that emit light within one quarter of the wavelength of an EUV period, emit coherently. In addition, when the bunch length is less than ¼ λ, the probability that an EUV proton is produced is higher.

Pulse bunching increases peak instantaneous charge density which ultimately reduces the laser threshold since the EUV light emitter is used solely to amplify the light. The system is advantageous because the electrons are bunched before they have been accelerated. When electrons have been accelerated, they are high energy, and, therefore, harder to move. By bunching the electrons before they have been accelerated, it is easier to move the electrons. Thus, the electrons are bunched at a first lower energy and light is generated when the electrons are at a second, higher energy. The advantage of bunching the electrons before acceleration is that a smaller accelerator is needed because the electrons are at a low energy when they are bunched. This leads to a reduced size, complexity, cost, and a simple, more reliable EUV source.

Electron guns typically generate pulses which are called macro pulses or macro bunches. The buncher is used to generate nano bunches. If radio frequency acceleration is used then the beam may also need to be broken down into micro bunches or micro pulses. Macro pulses are typically in the microsecond range, and include continuous streams of electrons. Micro pulses have a length which is dependant on the RF frequency used to accelerate the electrons. Typically these are greater than 3 mm long in length scale. The buncher may be used to generate nano pulses with frequency components as low as 13.5 nanometers long. The buncher can also produce nano pulses with frequency components that are 10 times and even up to and including 20 times longer then 13.5 nanometers long.

Using a transition radiation stack to extract light from the bunched beam is advantageous because high energy electrons incident upon a dielectric interface can emit EUV. When another photon is present, stimulated emission can occur, and the probability of getting a second photon is much greater. Stimulated transition radiation therefore produces a lasing effect. Electrons can also interact with the EUV radiation field, which can lead to further micro-pulse bunching, which further increases gain.

The combination of bunching and transition radiation further increases the probability of stimulated emission (laser action). Stimulated emission occurs when electrons arrive at the light emitting stage at the same time. Therefore, the creation of micro-bunches can radically increase the available light as a result of the stimulated emission processes.

Compton scattering is advantageous because it is more efficient than simply adding the wiggler radiation field and electron beam. By generating the field at an oblique angle and at a harmonic of the wiggler frequency, it is possible to produce temporally coherent pulses. A small energy modulation is imposed on the electron beam by its interaction with a seed laser. The resulting energy modulation is converted into a longitudinal density modulation as the electron beam traverses a magnetic dispersion, in a second undulator, which is tuned to the nth harmonic of the seed frequency. The micro-bunched electron beam emits coherent radiation at the harmonic frequency, which is then amplified in the radiator until saturation is reached (i.e., high gain generation).

In one embodiment, the oblique laser has a near diffraction limited spot in one direction, which lessens the emittance and energy spread requirements of the electron beam. In another embodiment, bunching may start from the spontaneous emission process with significantly higher wiggler powers. An optical delay line between the wiggler and electron gun may be required with an interval that matches the micro-pulse period of the beam.

Compton backscattering is also a mechanism for generating EUV photons from a relativistic electron beam. For example, scattering a CO2 laser (10.6 μm) at 34.8 degrees from a 7 MeV beam generates 13.5 nm light at an angle from the beam of 0.042 degrees.

The foregoing description with attached drawings is only illustrative of possible embodiments of the described method and should only be construed as such. Other persons of ordinary skill in the art will realize that many other specific embodiments are possible that fall within the scope and spirit of the present idea. The scope of the invention is indicated by the following claims rather than by the foregoing description. Any and all modifications which come within the meaning and range of equivalency of the following claims are to be considered within their scope.

What is claimed is:

1. A method to emit electromagnetic radiation comprising:
providing an electron beam;
modulating the density of electrons in the electron beam;
accelerating the electron beam;
compensating for electron dispersion in the electron beam; and
generating radiation from the electron beam.

2. The method of claim 1, wherein modulating the density of electrons in the electron beam comprises bunching the electrons in the electron beam.

3. The method of claim 2, wherein bunching the plurality of electrons comprises pulse bunching at a plurality of length scales, the plurality of electrons and bunches having at least one frequency component such that the plurality of electrons and bunches radiate coherently.

4. The method of claim 1, wherein the density of electrons is modulated with an optical buncher.

5. The method of claim 4, wherein the optical buncher comprises at least a first laser, the first laser generating a first beam and a second beam, the second beam being a back-scattered electromagnetic wave.

6. The method of claim 5, wherein the first beam is an oblique counter-propagating wiggler relative to the electron beam and wherein the second beam is a back-scattered electromagnetic wave.

7. The method of claim 5, wherein the second beam is at a harmonic of the first beam.

8. The method of claim 5, further comprising a second laser, the second laser generating a third beam, the third beam seeding with the second beam of the first laser.

9. The method of claim 4, wherein the electron beam is modulated to a contain frequency components at a harmonic of the optical buncher wavelength.

10. The method of claim 1, further comprising accelerating the electrons before bunching the electrons to an energy level of at most 12 MeV.

11. The method of claim 1, wherein electron dispersion is compensated before the electron bunches are accelerated.

12. The method of claim 1, wherein electron dispersion is compensated after the electron bunches are accelerated.

13. The method of claim 1, wherein a radio frequency linear accelerator both accelerates and electron beam and compensates for electron dispersion in the electron beam.

14. The method of claim 1, wherein radiation is generated using transition radiation.

15. The method of claim 1, wherein radiation is generated using Compton or Thompson back-scattered radiation.

16. The method of claim 1, wherein the radiation generated from the electron beam is extreme ultraviolet radiation, soft x-ray radiation or ultraviolet radiation.

17. An electromagnetic radiation source comprising:
an electron gun, the electron gun emitting an electron beam;
a buncher, the electron beam passing through the buncher to modulate the density of electrons in the electron beam;
an accelerator, the accelerator accelerating the electron beam;
an electron dispersion compensator, the electron dispersion compensator compensating for dispersion of electrons in the bunched electron beam; and
a radiation generator, the generator generating radiation from the electron beam.

18. The radiation source of claim 17, wherein the electron dispersion compensator compensates for dispersion of electrons before the beam is accelerated.

19. The radiation source of claim 17, wherein the electron dispersion compensator compensates for dispersion of electron beams after the beam is accelerated.

20. The radiation source of claim 17, wherein the accelerator is a radio frequency linear accelerator that includes the electron dispersion compensator.

21. The radiation source of claim 17, wherein the buncher is an optical wiggler.

22. The radiation source of claim 21, wherein the optical wiggler comprises at least a first laser, the first laser generating a first beam and a second beam, the second beam being a back-scattered electromagnetic wave.

23. The method of claim 22, wherein the first beam is an oblique counter-propagating wiggler relative to the electron beam and wherein the second beam is a back-scattered electromagnetic wave.

24. The method of claim 22, wherein the second beam is at a harmonic of the first beam.

25. The method of claim 22, further comprising a second laser, the second laser generating a third beam, the third beam seeding with the second beam of the first laser.

26. The method of claim 21, wherein the electron beam is modulated to a contain frequency components at a harmonic of the optical wiggler wavelength.

27. The radiation source of claim 17, wherein the generator is a transition radiation stack.

28. The radiation source of claim 17, wherein the generator is an optical buncher that uses Thompson or Compton back-scattering.

29. The radiation source of claim 17, wherein the radiation generator generates extreme ultraviolet radiation, soft x-ray radiation or ultraviolet radiation.

30. The radiation source of claim 17, further comprising a pre-accelerator, the pre-accelerator accelerating the electron beam to at most 12 MeV before the electron beam passes through the buncher.
